(19) United States
(12) Patent Application Publication
(10) Pub. No.: US 2012/0232330 A1
(43) Pub. Date: Sep. 13, 2012

(54) Device for Administering Drugs and for Influencing the Effects of Drugs

(76) Inventor: Bjarne Geiges, Muenchen (DE)

(21) Appl. No.: 13/502,050

(22) PCT Filed: Oct. 13, 2010
(86) PCT No.: PCT/EP2010/006271

§ 371 Date: Jun. 4, 2012

(30) Foreign Application Priority Data
Oct. 13, 2009 (DE) 10 2009 049 175.9

(51) Int. Cl. A61M 37/00; H01R 43/00

(52) U.S. Cl. 600/13; 29/825

(57) ABSTRACT

A device has a voltage source, an electric conductor connected to an electric pole of the voltage source and acting as an electrode, and a substrate arranged in an electromagnetic field which is formed in the vicinity of the electric conductor when the voltage source is active. The substrate is used to receive drugs.

### BACKGROUND AND SUMMARY OF THE INVENTION

The invention relates to a device for administering drugs and for influencing the effects of drugs.

In connection with the administration of drugs, the principal problem is that they are generally applied without or with only insufficient localising measures. Therefore, not only an affected body region will come in contact with the active substances, but in fact, healthy body cells will also be affected and possibly permanently damaged by the administered substances.

Another problem affecting the effectivity of drugs is associated with the fact that the cell membranes to be penetrated and the biochemical protective covers of cells constitute an obstacle to the drugs. For example, bacteria may become resistant to antibiotics in this way, and cancer cells may survive despite of the application of cytostatics. It may also be problematic to apply growth-enhancing substances or other cellular active substances so that the cell membranes are penetrated.

But even if the active substance has reached the interior of the cell, there may be intracellular reaction impediments which are, in many cases, simply associated with the kinetics of the required chemical reaction, i.e. particularly with the absence of the activation energy required for the reaction.

The object of the invention is to address the named problems individually or collectively, in particular, to concentrate drugs locally, to enhance the penetration of cell membranes, and to influence the kinetics of the intracellular reaction.

Said object is solved by the features of the independent claim.

Advantageous embodiments of the invention are described in the dependent claims.

The invention relates to a device comprising a voltage source, an electric conductor connected to an electric pole of the voltage source and acting as an electrode, and a substrate arranged in the electromagnetic field which forms in the vicinity of the electric conductor when the voltage source is active, wherein the substrate serves to accommodate drugs. Such a device can be placed in the direct vicinity of the affected body region. With the voltage source or the electromagnetic field formed in the vicinity of the electric conductor the release of the drugs accommodated by the substrate can be accomplished or enhanced. More specifically, the term “substrate” used here can, also be replaced by the term “matrix”. Body regions located away from the device placed in a localised manner will not be or will only scantly be reached by the locally applied drugs. Further, the presence of an electric field gradient will have the effect that the penetration of the drugs through the cell membranes is facilitated. The electric field may also cause a concentration of ionic messenger substances (e.g., Ca²⁺), so that the penetration through the membrane pore opening is thereby also enhanced.

It is particularly preferred that a device for influencing an electric voltage generated by the voltage source is provided. In this way the release of drugs from the substrate can be controlled. The device may then, for example, be designed so that an increased drug release will take place when the electric voltage is increased.

Preferably, the electric conductor connected to the electric pole is a metal wire. In principle, any electric conductor can be used. A metal wire, particularly a stainless steel wire, however, constitutes a useful possibility to transfer the electric potentials generated by the voltage source.

Usefully, it is contemplated that the metal wire carries the substrate. Aside from the electric properties of the metal wire required for the invention, this also provides a safe and flexible possibility to accommodate the substrate containing the drugs. The substrate formed of an insoluble matrix such as PMMA (polymethyl methacrylate) and having the form of, for example, spheres or ellipsoids may be accommodated by a carrier, usually a steel wire, similar to a pearl necklace, which is implanted in the area of an infection, for example, a bone infection.

In connection with the present invention, it is particularly useful that the voltage source comprises a transducer which generates an electric voltage in the presence of an external magnetic field. An external alternating magnetic field can generate an electric voltage in a transducer which is, in particular, realised by a coil. Thus, the variation of the external magnetic field can also induce a variation of the electric voltage and thus of the release rate of the drug. Further, the reaction kinetics can be influenced by the alternating magnetic field in such a way that the reaction of the cell substances with the drugs can already take place at a low activation energy.

It may also be contemplated that the voltage source comprises a battery. Such a battery may, in particular, be provided in addition to a transducer which generates an alternating electric field, namely for superimposing the alternating voltage with a direct voltage component.

The present invention is further developed in a particularly useful way in that an at least partly electrically conductive implant is electrically coupled to a pole of the voltage source. The invention can be deployed in an advantageous manner particularly in the area of endoprostheses and osteosynthesis means. On the one hand, it is known that the presence of low-frequency alternating magnetic fields and the electric stimulation of implants caused thereby by means of a transducer have a positive influence on the encouragement and the differentiation of the tissue growth. Exemplary systems are described in the following documents: DE 199 28 449 C1, DE 10 2004 024 473 A1, DE 10 2006 018 191 B4, DE 10 2007 049 542 A1, and DE 10 2007 063 027 A1. In the devices and methods shown therein, low-frequency alternating electric potentials are generated in implants by exposing an affected body part to an alternating magnetic field. For a long time, it has been shown in numerous clinical applications of this technology to chronically therapy-resistant, in most cases infected bone defects, cysts, and tumour metastases as well in clinical experimental studies that an optimum healing effect is achieved by using implants as sources of extremely low-frequency sinusoidal alternating electric potentials in the bone region abutting on the support metal. The same applies to endoprostheses. The technology for transmission functions according to the transformer principle: the injured or affected body region is flooded with an extremely low-frequency sinusoidal magnetic field having a frequency of approximately 1 to 100 Hz—preferably of 4 to 20 Hz—and a magnetic flux density of 0.5 to 5 mT (5 to 50 Gauss) generated by a waveform generator in one or more—primary—external current coils into which the body part provided with the implants is introduced. These extremely low-frequency electromagnetic fields permeate the tissue including any present clothing and/or a cast, as well as the non-magnetic (austenitic) support metals of the implants largely without loss. A—secondary—coil arrangement, the so-called transducer, is implanted in electric contact with these. In this way, the electrical potentials induced in the transducer are rendered effective in the area of the bone lesion as well as generally in the tissues adjacent to the implants. With the aid of the invention, it is now realised that on the one hand, the implants are electrified in the described manner. On the other hand, a local administration of drugs using the same means is enabled.

According to a specific embodiment of the invention, it is contemplated that a tooth-jaw bridge is provided which at least partly forms or carries the substrate and which further contains the voltage source as well as the electrodes interacting with the voltage source. Such a tooth-jaw bridge may, owing to its bone-generating effect, replace the complicated mechanical callus distraction of the alveolar jaw area. Further, infected tooth prostheses in the form of the enossal jaw implants—commonly referred to as peri-implantitis—can be treated successfully. The electrodes of the tooth-jaw bridge are, in this case, preferably arranged on the buccal and lingual sides. Direct or alternating voltages in the range from 500 to 700 mV are achievable between the electrodes by means of which the bone growth is stimulated and a strongly antibiotic effect is developed. Bacteria biofilms are detached from their carrier, for example, a dental prosthesis formed of a titanium alloy, and its virulence due to which approximately 20 to 30 percent of all dental prostheses are still presently lost is brought to an end. Aside from the treatment of the peri-implantitis, this can also be prevented by the specific embodiment of the invention comprising the tooth-jaw bridge. At the tooth gap in the jaw, bone growth is stimulated by the tooth-jaw bridge and the electro-osteotherapy thereby enabled so that the jaw is prepared for a later implantation. At the same time, an infection prior to implantation is excluded by the antibacterial effect of the tooth-jaw bridge.

In this connection, it is particularly useful that the tooth-jaw bridge contains two bridge abutments and a bridge platform, that the voltage source is located in the bridge platform, and that an electrode connected to a respective pole of the voltage source as well as the substrate directly contacting gingiva and mucosa are arranged in the bridge abutments, respectively.

According to a preferred embodiment of the device for jaw treatment, it is contemplated that at least one component carrying the substrate and the electrode interacting with the voltage source is manufactured with the aid of a plastic impression of a body part to be contacted. Due to the strongly rippled and profiled surfaces of the tooth and jaw region, establishing a sufficient contact between the electrodes and the body parts to be contacted using conventional tools is problematic. For example, a support plate as used in the osteosynthesis of fractures of long hollow bones would lose stimulating effectiveness due to the lack of electric contact. Also in regard to the release of drugs, a simple plate would not be ideal, as the release depends on the size of the surface of the substrate relative to the dimensions of the defect area. These problems are solved by the use of a plastic impression. Preferably, buccal as well as lingual plastic impressions are prepared onto which then an electrically conductive, porous and compressible layer is finally applied. This layer forms the substrate for the drugs. It is possible to apply a conducting surface layer which particularly serves to contact the connections brought up from the voltage source onto the impressions prior to the application of the electrically conductive, porous, and compressible layer. The plastic impressions are preferably held by a bracket which might also serve as a carrier for the pick-up coil and/or the battery and/or a direct current/alternating current generator. The electrically conductive, porous, and compressible layers may wholly or partly consist of an electrically conductive plastic material, for example, polymers which can be converted into an electrically conductive form by doping and/or coating.

It may also be contemplated that at least one component forming the substrate and the electrode interacting with the voltage source is formed of an at least partly conductive and absorptive material. The fabrication of plastic impressions requires a certain effort. This can be avoided by manufacturing the entirety of the electrodes from a sponge-like elastic material having a good electric conductivity. The same materials come into consideration as the ones used for the electrically conductive, porous and compressible coating of the plastic impressions. While in case of the plastic impressions, the electrically conductive structures are preferably applied exclusively where an electric contact to the body is to be established and also where the drugs are to be released, it is reasonable in case of the electrodes consisting entirely of a porous material to electrically insulate the buccal and lingual areas and also to prevent the release of drugs in these places by converting the surfaces into a liquid-impermeable form there, for example, by coating.

According to a preferred embodiment of the invention, it is contemplated that the substrate at least partly consists of polymethyl methacrylate. Polymethyl methacrylate (PMMA) is a biologically safe active substance which is, furthermore, suitable to accommodate drugs and to release them at the location of the illness.

It is particularly preferred that the substrate at least partly consists of a magnesium-calcium alloy or a magnesium-zinc-calcium alloy. One drawback of the use of PMMA becomes obvious when it is explanted from the tissue in which the substrate chain has become more or less ingrown so that a larger surgical operation is required to remove it. This drawback can, according to the invention, be avoided by the use of substrates consisting of a biocompatible material which is soluble in the tissue. A particularly suitable substrate for this purpose is magnesium in alloys containing calcium and/or zinc and/or also lithium and/or rare earths. Due to its direct affinity with calcium phosphate, the main component of the hard bone substance, the alloy of magnesium and calcium is particularly suitable for the antibiotic treatment of infected bone areas. A magnesium alloy may also serve as a carrier for bone growth factors, for example BMP (bone morphogenetic protein) or TGF Beta (transforming growth factor beta). A further advantage of magnesium as a drug carrier in the arrangement according to the invention is, its electric conductivity which at 22.6 S/m, ranges between aluminium (36.6 S/m) and brass (15.5 S/m).

The invention further consists of a system comprising a device according to the invention to be attached to or to be introduced into a human body and a device for generating a magnetic field outside of the body.

Further, the invention relates to a method for producing a device according to the invention, wherein, in the course of the production of the device, at least one component carrying the substrate and the electrode interacting with the voltage source is produced by means of a plastic impression of a body part to be contacted.

The invention was primarily described in connection with implants above. However, it is not limited to this. Another useful application is, for example, a wound dressing comprising an electrode network and an antibiotic, particularly gentamyzin, wherein a collagen mesh forms the substrate.

The invention is based on the finding that with consistent technology, the principles of the electro-osteotherapy can be applied and, at the same time, that the issues in connection with the administration of drugs described in the introduction can be addressed. Drugs can be administrated locally, the penetration of cell membranes is facilitated, and the kinetics of the intracellular reaction is accelerated. The presence of magnetic fields can also be used in another way, namely in which the active ingredients are coupled to a paramagnetic carrier. In this case, the carrier contains nanoparticles having the required magnetic properties. By placing the area of the patient where the active substances are to be concentrated in a spatially variable magnetic field, i.e., what is commonly referred to as a gradient magnetic field, so that the substances are concentrated in the area of the cells to be treated, for example, in the area of a tumour, other body regions may remain unaffected by the active substances to be administered. The active substances are thus applied locally in the vicinity of the body region to be treated, and they are then concentrated in the affected tissue due to the magnetic gradient fields. This method is also referred as “magnetofection”.

### BRIEF DESCRIPTION OF THE DRAWINGS

The invention will now be explained by way of example with the aid of particularly preferred embodiments with reference to the accompanying drawings in which:

FIG. 1 shows an embodiment of a device according to the invention;

FIG. 2 shows a further embodiment of a device according to the invention;

FIG. 3 shows an embodiment of the present invention coupled to an implant;

FIG. 4 shows an embodiment of the present invention coupled to another implant;

FIG. 5 shows a further embodiment of the present invention;

FIG. 6 shows an embodiment of the present invention formed as a tooth-jaw bridge;

FIG. 7 shows a side view of the embodiment according to FIG. 6;

FIG. 8 shows another side view of the embodiment according to FIG. 6;

FIG. 9 shows a detail of the embodiment according to FIG. 6;

FIG. 10 shows an embodiment of a tooth spacer comprising an electrode;

FIG. 11 shows another embodiment of a tooth spacer comprising an electrode;

FIG. 12 shows another embodiment of a tooth spacer comprising an electrode;

FIG. 13 shows a specific embodiment of a device according to the invention to be used in the jaw area; and

FIG. 14 shows another embodiment of a device according to the invention to be used in the jaw area.

### DETAILED DESCRIPTION OF THE DRAWINGS

In the following description of the drawings identical numerals designate the same or comparable components.

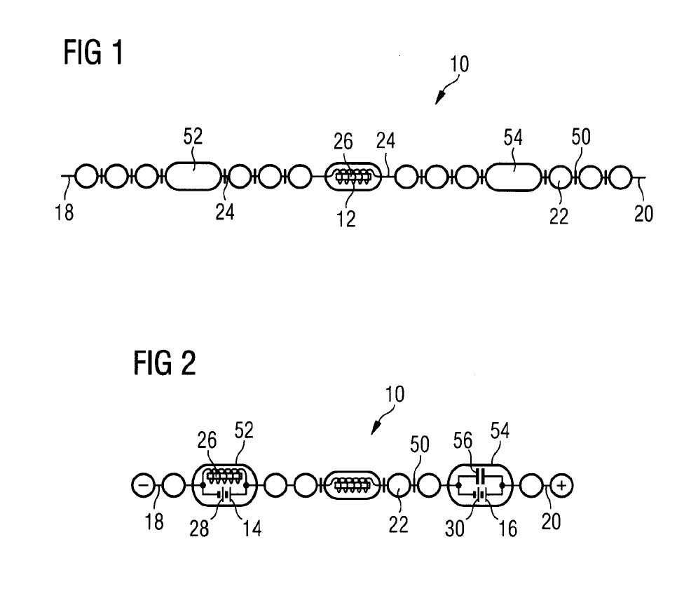

FIG. 1 shows an embodiment of a device according to the invention. The device 10 comprises a voltage source 12 which is realised as a transducer 26 here. The transducer 26 may be a coil winding on a magnetically soft core. At the voltage source 12, metal wires 24 acting as electrodes 18, 20 are connected to the respective poles. Die metal wires 24 carry spheres constituting a substrate 22 for drugs. The substrate 22 may also be formed by elements having another shape. The individual elements of the substrate 22 are separated from each other by optionally provided spacers 50 of a conductive or non-conductive material, i.e. spacers in the form of platelets made of the same material, of the wire carrying the substrate spheres (ellipsoids). On the metal wires 24, there are accommodation devices 52, 54 in which further components can be disposed, for example, further voltage sources or other electric elements.

If the device 10 according to FIG. 1 is placed in an alternating magnetic field, an alternating voltage is located between the electrodes 18, 20. Owing to the electromagnetic field, the release of the drug from the substrate 22 can be stimulated. By varying the external magnetic field and with it the electric field the rate of the drug release can be controlled. As a substrate, polymethyl methacrylate (PMMA) is, in particular, to be considered. Resorbable carriers may also be used. For example, collagen, calcium sulphate, and magnesium can be used for antibiotics.

FIG. 2 shows another embodiment of a device according to the invention. The arrangement shown here is comparable to the one in FIG. 1. In addition, electrical components can be seen which are arranged inside of the accommodation devices 52, 54. A battery 28 imprinting a direct voltage component on the alternating voltage generated by the external alternating magnetic field is located inside of the accommodation devices 52. The battery 28 may be connected in parallel to another transducer 26 as shown in the present example. The accommodation device 54 also contains a battery 30. In the present example, it is connected in parallel to a capacitor 56. Owing to the combination of a battery generating a direct voltage and the alternating electric field stimulated by an external magnetic field, the alternating voltage may be varied by directly influencing the external magnetic field, and at the same time, tissue surrounding the electrodes 18, 20 may be specified as an anode and a cathode which may influence the release of the drug and the electrodes 18, 20. The parallel connection of a capacitor 56 to a direct voltage source 30 permits the application of a direct voltage with a super-imposed alternating voltage component, the amplitude of which depends on the size of the capacitor. For example, electrolytic capacitors or supercaps may be used as capacitors. It is also possible to operate the device with pure direct voltage, i.e. either using a rectified voltage generated by a transducer or only on the basis of a battery. The arrangement will only become effective in the presence of an upstream transducer transmitting an alternating electric potential to the wire electrode which bridges it by means of the capacitor connected in parallel to the battery.

Exemplary operating modes may be characterised as follows:
A pure electromagnetically induced alternating voltage having a frequency of 10 to 20 Hz is used.
A pure direct voltage from an integrated battery of 700 to 800 mV is used.
By combining a direct voltage source and an alternating voltage source, a permanent direct voltage of 200 to 400 mV is generated and is temporarily or permanently superimposed by an alternating voltage having an amplitude of approximately 500 mV.

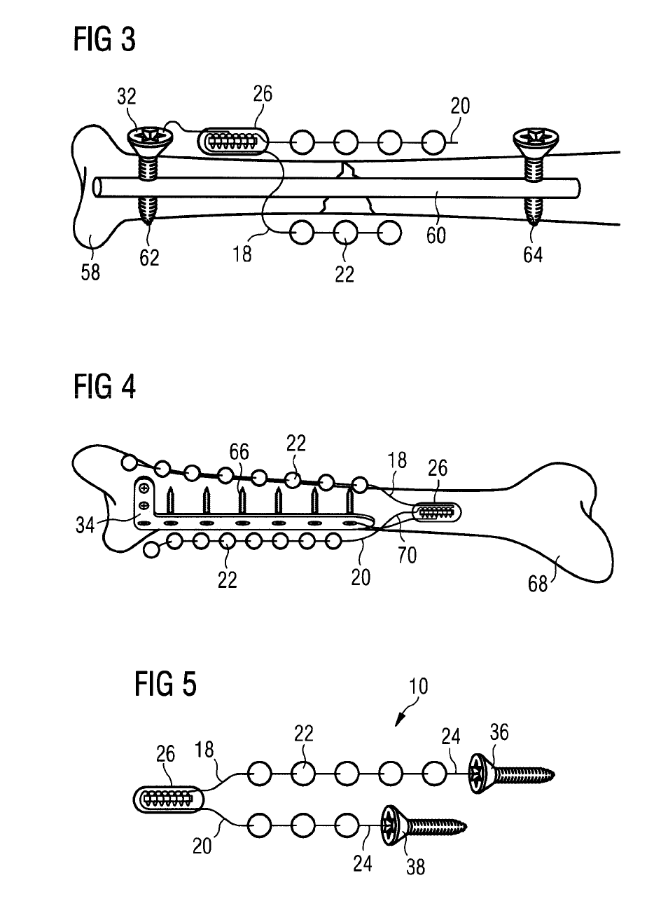

FIG. 3 shows an embodiment of the present invention coupled to an implant. A bone 58 is furnished with a marrow nail 60. The marrow nail 60 is fixed by locking screws 62, 64. These implants, in the present example particularly implant 32, namely one of the locking screws, is connected to a pole of a transducer 26. Further electrodes 18 carrying the drug substrates 22 mentioned in connection with FIGS. 1 and 2 are located at the transducer 26. In this case the coupling to the transducer 26 may be implemented so that the electrodes 18, 20 have the same potential or inverse potentials, according to requirements. With the shown arrangement, it is therefore possible to deploy the implant 32 within the framework of the electro-osteotherapy and, at the same time, to use the electric voltage generated by the transducer 26 with regard to the release of drugs.

FIG. 4 shows an embodiment of the present invention coupled to another implant. The implant 34 shown here is a bone support plate fixed to a bone 68 by a plurality of screws 66. A voltage source, here, in particular, once again a transducer 26, is, on the one hand, connected to the bone support plate via an electric line 70. Furthermore, two electrodes 18, 20 carrying substrates 22 with drugs are connected. Once again, the electric connection of the components at the transducer 26 may be realised in different ways. For example, the bone support plate may have an inverse potential with regard to the two electrodes 18, 20. It is also feasible to connect the bone support plate to a central pickup of the coil in the transducer 26, while the two electrodes 18, 20 form the two inversely charged electrodes. The bone support plate may also have the same potential as one of the inversely poled electrodes 18, 20.

FIG. 5 shows another embodiment of the present invention. In the device 10 shown here, the transducer 26 is connected to respectively one screw 36, 38 via the electrodes 18, 20. The metal wires 24 leading to the screws 36, 38 in turn carry the substrate 22. Here the screws 36, 38 form the electrodes 18, 20 having inverse potentials. For example, screws coated with titanium-niobium-oxynitride may be used as the screws 36, 38.

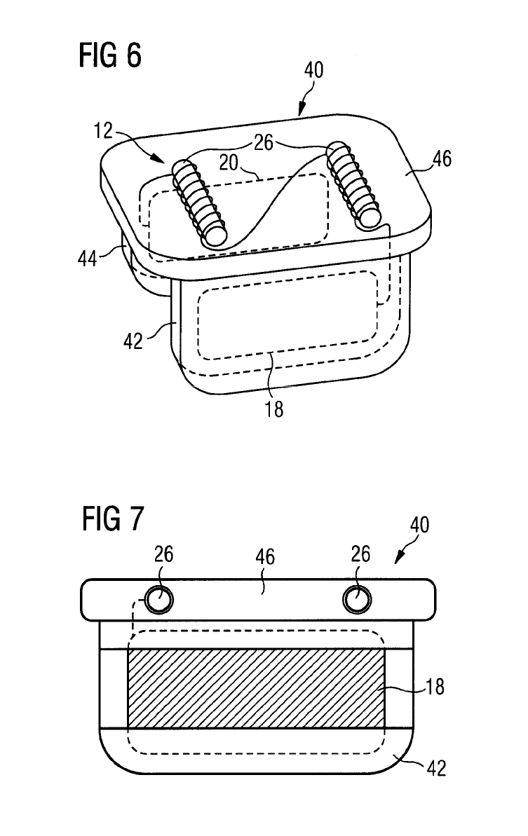

FIG. 6 shows an embodiment of the present invention formed as a tooth-jaw bridge. The tooth-jaw bridge 40 shown here serves the formation of the alveolar jaw bone. The tooth-jaw bridge 40 shown here comprises two bridge abutments 42, 44 and a bridge platform 46. In the bridge platform 46, the voltage source 12 is arranged, which is here, by way of example, once again a transducer 26 comprising two coils electrically connected in series. In the bridge abutments 42, 44, the electrodes 18, 20 are arranged, whereby the electrode 18 is located in the bridge abutment 42, and the electrode 20 is located in the bridge abutment 44. The induction coils of the transducer 46 are, in the present example, wound over flat iron cores and then covered by Teflon or epoxy resin. Similarly, the electrodes 18, 20 may be completely or partly coated.

FIG. 7 shows a side view of the embodiment according to FIG. 6. One of the bridge abutments 42 with the electrode 18 located therein can be seen. Further, the bridge platform 46 with the transducer 26 embedded therein can be seen.

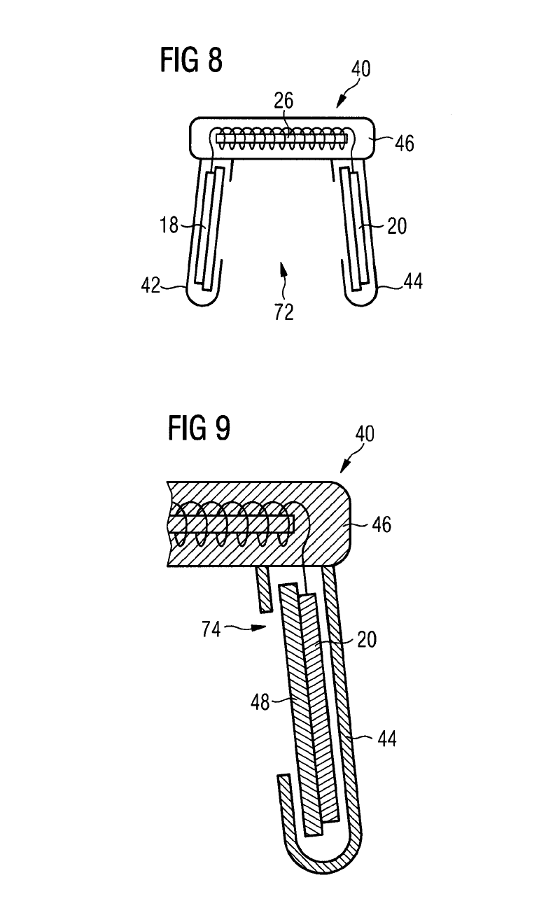

FIG. 8 shows another side view of the embodiment according to FIG. 6. In this illustration both bridge abutments 42, 44 with the respective electrodes 18, 20, as well as the bridge platform 46 with the transducer 26 can be seen. Area 72 into which the jaw bone is to grow is also visible.

FIG. 9 shows a detail of the embodiment according to FIG. 6. In addition to the electrode 20 in the shown bridge abutment 44, a substrate 48 can be seen here which is arranged adjacent to the electrode 20 and for which an opening 74 through which the drug accommodated by the substrate 48 can be released into the cavity 74 is provided on the side of the bridge abutment 44 facing the cavity.

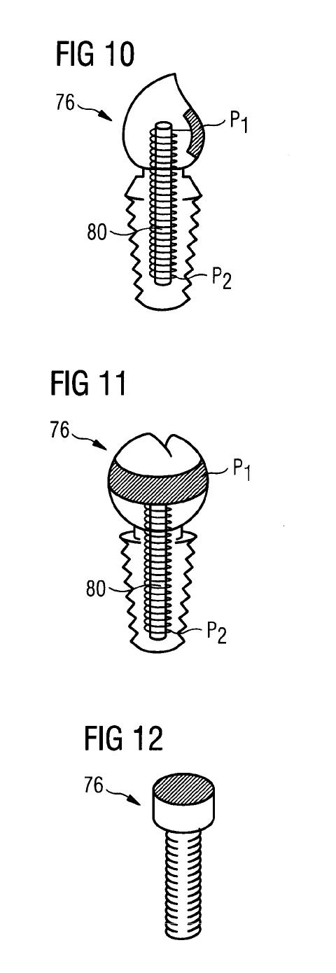

FIG. 10 shows an embodiment of a tooth spacer comprising an electrode. Here an example of a tooth spacer 76 to be screwed into the thread of a carrier of a dental prosthesis implanted into the jaw bone is shown. In its interior, the tooth spacer 76 contains a transducer 80 which creates inverse potentials P₁ and P₂ in two different areas of the tooth spacer 76 which are electrically insulated from each other. To stimulate the bone growth, the transducer 80 is excited by the same external alternating magnetic field for generating an a electric voltage which also excites the transducer in the tooth-jaw bridge by which ultimately the drug release from the substrate 48 (see FIG. 9) can be stimulated or controlled.

FIGS. 11 and 12 show two further constructive examples of tooth spacers 76 comprising an electrode, wherein in FIG. 12 the internal transducer 80 can be seen again.

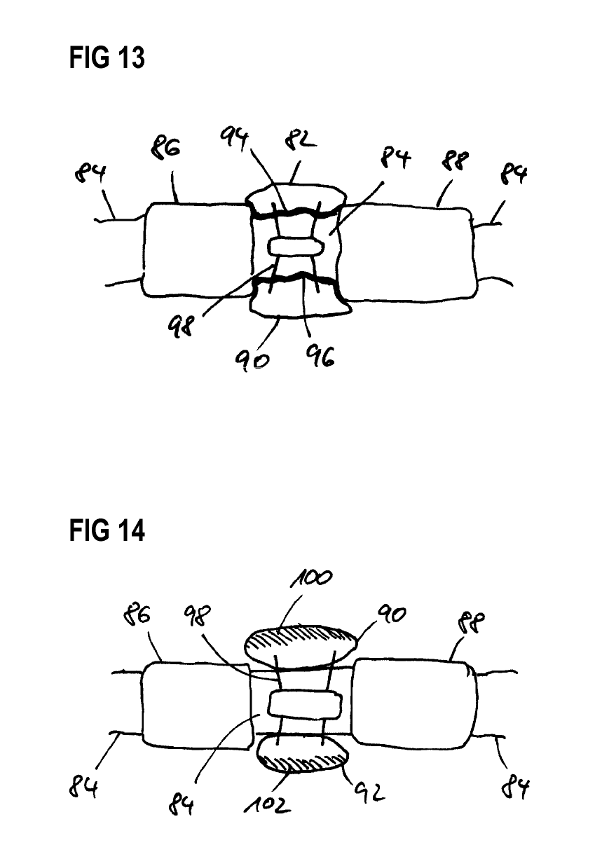

FIG. 13 shows a specific embodiment of a device according to the invention to be used in the jaw area. A part of a jaw 84 comprising two intact teeth 86, 88 is shown. Two components 82, 90 arranged on the lingual and buccal sides of the jaw precisely abut on the jaw 84. This can be achieved by manufacturing the components 82, 90 by means of a plastic impression of the jaw 84 to be contacted. After the production of the plastic impression a porous, electrically conductive, and compressible layer 94, 96 is finally applied to the components 82, 90 manufactured with the aid of the plastic impression, wherein these layers 94, 96 are to cover, in particular, the area of the components 82, 90 facing the jaw 84. The sides of the components 82, 90 facing away from the jaw are not to be electrically conductive and are not to have any absorption capacity for drugs which is, in case of plastic impressions, generally ensured from the beginning. Prior to the application of the compressible, electrically conductive, and porous layers 94, 96 to the components 82, 90, a conventional electric coating may first be applied to the components 82, 90 which will then carry the electrically conductive, porous and compressible layers. This intermediate coating primarily serves to connect the voltage source. The components 82, 90 will then be held by a bracket 98. The bracket also carries the transducer and/or battery and/or the direct current/alternating current generator, and it supplies the electric voltages to the components 82, 90 coated with electrodes. In the present case in which a tooth gap is present between two intact teeth 86, 88, there is sufficient space between the teeth to accommodate the bracket 98 together with the components 82, 96. If a device according to the invention is to be implanted to heal an already existing peri-implantitis at an already implanted implant, the bracket will usually rest on the implant like a nightguard.

FIG. 14 shows another embodiment of a device according to the invention to be used in the jaw area. The device shown here is, in large part, similar to the one according to FIG. 13. However, the components 90, 92 according to FIG. 14 differ from the components 82, 90 according to FIG. 13. Namely, the components 90, 92 according to FIG. 14 are entirely formed of a porous, electrically conductive, and compressible or elastic material. The hatched areas 100, 102 are designed so that they are electrically insulated and liquid-impermeable, for example by means of coatings. Owing to the effect of the bracket 98, the components 90, 92 which are, in particular, designed as ellipsoids, cling to the jaw 84, and therefore, they ensure the good electric contact required for the success of the antibiotic treatment stimulating the bone growth.

The features of the invention disclosed in the above description, in the drawings as well as in the claims may be important for the realisation of the invention individually as well as in any combination.

### LIST OF NUMERALS

10 device

12 voltage source

14 voltage source

16 voltage source

18 electrodes

20 electrodes

22 substrate

24 metal wires

26 transducer

28 battery

30 battery

32 implant

34 implant

36 screw

38 screw

40 tooth-jaw bridge

42 bridge abutment

44 bridge abutment

46 bridge platform

48 substrate

50 spacer

52 accommodation device

54 accommodation device

56 capacitor

58 bone

60 marrow nail

62 locking screw

64 locking screw

66 screws

68 bone

70 electric line

72 cavity

74 opening

76 tooth spacer

80 transducer

82 component

84 jaw

86 tooth

88 tooth

90 component

92 component

94 layer

96 layer

98 bracket

100 insulated area

102 insulated area

1.-15. (canceled)

16. A device comprising:
a voltage source,
an electric conductor connected to an electric pole of the voltage source and acting as an electrode; and
a substrate arranged in an electromagnetic field which forms in the vicinity of the electric conductor when the voltage source is active, wherein the substrate serves the accommodation of drugs.

17. The device according to claim 16, wherein a device for influencing an electric voltage generated by the voltage source is provided.

18. The device according to claim 16, wherein the electric conductor connected to the electric pole is a metal wire.

19. The device according to claim 18, wherein the metal wire carries the substrate.

20. The device according to claim 16, wherein the voltage source comprises a transducer generating an electric voltage in the presence of an external magnetic field.

21. The device according to claim 16, wherein the voltage source comprises a battery.

22. The device according to claim 16, wherein an at least partly electrically conductive implant is electrically coupled to a pole of the voltage source.

23. The device according to claim 16, wherein a tooth-jaw bridge is provided which at least partly forms the substrate or carries the substrate and which further contains the voltage source as well as the electrode interacting with the voltage source.

24. The device according to claim 23, wherein the tooth-jaw bridge contains two bridge abutments and a bridge platform, in that the voltage source is located in the bridge platform, and in that an electrode connected to a respective pole of the voltage source as well as the substrate are arranged in the bridge abutments, respectively.

25. The device according to claim 23, wherein at least one component carrying the substrate and the electrode interacting with the voltage source is produced with the aid of a plastic impression of a body part to be contacted.

26. The device according to claim 23, wherein at least one component forming the substrate and the electrode interacting with the voltage source is formed by an at least partly conductive and absorptive material.

27. The device according to claim 16, wherein the substrate is made at least partly of polymethyl methacrylate.

28. The device according to claim 16, wherein the substrate is made, at least partly, of a magnesium-calcium alloy or a magnesium-zinc-calcium alloy.

29. The device according to claim 17, wherein the electric conductor connected to the electric pole is a metal wire.

30. A system, comprising:
a voltage source,
an electric conductor connected to an electric pole of the voltage source and acting as an electrode;
a substrate arranged in an electromagnetic field which forms in the vicinity of the electric conductor when the voltage source is active, wherein the substrate serves the accommodation of drugs attached to or to be introduced into a human body; and
a device for generating the electromagnetic field outside of the body.

31. A method for producing a device comprising a voltage source, an electric conductor connected to an electric pole of the voltage source and acting as an electrode, and a substrate arranged in an electromagnetic field forming in a vicinity of the electric conductor when the voltage source is active, wherein the substrate serves the accommodation of drugs, wherein, in the course of the production of the device, at least one component carrying the substrate and the electrode interacting with the voltage source is manufactured with the aid of a plastic impression of a body part to be contacted.
